(19) United States
(12) Patent Application Publication
(10) Pub. No.: US 2006/0063720 A1
(43) Pub. Date: Mar. 23, 2006

(54) Oxaliplatin solution concentrate

(76) Inventors: Edgar Schridde, Hannover (DE); Bernd Merbach, Burgwedel (DE); Stefan Peter Gimmel, Hannover (DE)

Correspondence Address: KENYON & KENYON LLP, ONE BROADWAY, NEW YORK, NY, 10004, US

(21) Appl. No.: 10/947,603

(22) Filed: Sep. 22, 2004

(51) Int. Cl. A61K 31/70; A61K 31/28

(52) U.S. Cl. 514/23; 514/492; 514/53; 514/59

(57) ABSTRACT

The invention relates to oxaliplatin-containing infusion solution concentrates that include a physiologically-compatible carbohydrate as the solubilizer. The solutions and solution concentrates according to the invention are distinguished in that they are highly stable when stored, easy to handle, and well-tolerated.

### FIELD OF THE INVENTION

The invention relates to pharmaceutical preparations of oxaliplatin (I) for parenteral administration. Oxaliplatin (cis-oxalato-(trans-1,2-cyclohexanediamine)-platinum(II); cis-oxalato-(1,2-cyclohexanediamine)-platinum(II), trans-1,2-diaminocyclohexane oxaliplatinum; CAS No. 61825-94-3, Mw 397.3; C₈H₁₄N₂O₄Pt) is a compound first described in 2001 (Pharmeuropa Vol. 13, No. 3, 2001, p. 585-588). It represents a platinum (II) complex having one equivalent trans-1,2-diaminocyclohexane and one equivalent oxalic acid, and has the following chemical structure:

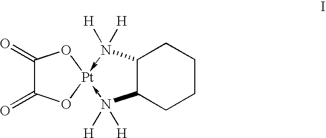

Oxaliplatin is a white, crystalline powder. It is soluble in water, scarcely soluble in methanol, and practically insoluble in ethanol. It is anti-neoplastic and is used by itself or in combination with 5-fuorouracil and/or folic acid in the therapy of metastatic colorectal cancer. The recommended dose in oxaliplatin therapy is 85 mg/m² body surface area. Like other platinum compounds, oxaliplatin may be used as a cytostatic in the therapy of a wide variety of cancers such as intestinal cancer, ovarian cancer, or cancer of the upper respiratory tract. Oxaliplatin is administered intravenously to treat a broad range of carcinomas, and thus the agent must be in the form of a solution.

### BACKGROUND OF THE INVENTION

Oxaliplatin solutions known from the related art that are required for parenteral administration immediately before being administered to the patient (mammal, preferably human) are reconstituted from a lyophilizate of oxaliplatin, or corresponding crystalline or amorphous solid preparations not manufactured by freeze-drying. However, there are substantial disadvantages associated with the use of such preparations. On the one hand, the procedure to manufacture the lyophilizate is complicated and expensive; on the other hand, reconstitution requires additional steps and represents an undesirable risk for personnel. In particular, the so-called to spray-back effect may occur when reconstituting the drug solutions from a dry substance, and this may cause contamination and endanger personnel. Accordingly, any contamination of personnel or inventory by the highly effective cytostatic must be prevented while producing the lyophilizate and reconstituting it. In addition, high demands are placed on the solvents used for reconstitution. They cannot be the conventional saline solutions for injection since this would cause the oxaliplatin complex to decompose. Serious difficulties in the oxaliplatin treatment may also arise from errors in handling this lyophilizate, such as deviations in the active ingredient concentration or microbial contamination of the solution. Given the many potential hazards and errors associated with the use of the lyophilizated agent for producing oxaliplatin solutions, it was therefore desirable to make available ready-to-use pharmaceutical solutions.

Oxaliplatin is not very stable in water, at least at concentrations below 1 mg/ml. In this context and in the rest of the description of the invention, “stability of the solution” is always to be understood as the stability of the oxaliplatin complex in solution, i.e., the long-term relative constancy of the concentration of the initial complex after it dissolves. The insufficient stability of oxaliplatin solutions in water results from the instability of the platinum complex itself, whose highly unstable ligands are exchangeable with other, stronger and more reactive nucleophiles, which causes the destruction of the initial complex. The type of additives used in platinum complex solutions is therefore highly crucial.

It is known, for example, that chloride anions cause oxaliplatin to decompose. This is why oxaliplatin lyophilizates cannot be reconstituted using saline solutions. Even the hydroxide anion from the solvent water is capable of substituting the ligands in a platinum complex. Unstable platinum complexes and hence oxaliplatin must therefore be stabilized in an aqueous solution. Diaquo DACH platinum (II) and diaquo DACH platinum dimer (III) are known examples of decomposition and reaction products of oxaliplatin (oxalato-DACH platinum) brought about by the presence of hydroxide anions.

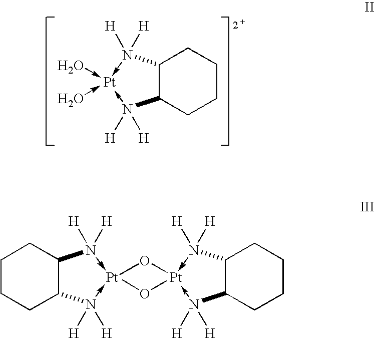

To stabilize the oxaliplatin complexes in aqueous solutions, the concentration and activity of the decomposing anions, such as the hydroxide anion, may be reduced in aqueous solvents. European Patent EP 0 774 963 therefore suggests increasing the concentration of oxaliplatin to at least 1 mg/ml so that, according to this document, an acidic pH value between 4.5 and 6 is achieved and the hydroxide anion concentration is lowered. However, when the examples from European Patent EP 0 774 963 were tested, only solutions having a pH above 6 were obtained.

The aqueous oxaliplatin solutions described in European Patent EP 0 774 963 are otherwise free of any acid or base, buffer or other additive since it was not possible to predict their interaction with the oxaliplatin complex and their effect on its stability. A problem with this approach is that oxaliplatin is scarcely soluble in water. The resulting potential of undesirable crystallization from supersaturated solutions not only decreases the agent concentration, but there is an immanent danger of undissolved particles triggering a potentially life-threatening embolism (vascular occlusion) upon injection and infusion.

A way to overcome this problem was illustrated in European Patent EP 1 207 875, where the solubility of the oxaliplatin complex in water is improved by adding 1,2-propanediol, glycerin, maltite, saccharose or inosite. Unfortunately, these additives have substantial disadvantages when creating injectable pharmaceutical preparations. They are all energy sources readily available as carbohydrates that may lead to the undesirable decompensation of the energy metabolism, especially in the case of the widespread age-related diabetes mellitus in the oxaliplatin therapeutic collective. In addition, inosite, for example, is a physiological, intracellular sugar, and its phosphate represents an essential component of a signal transduction cascade. Inosite is administered orally and intravenously in experimental therapy as an additional maturation promoter for premature babies having immature organs. An undesirable potential for neurological side effects also exists. Beyond the cited therapies, there is no approved or known use of inosite for injectable parenteral forms of administration.

The other hydroxy compounds presented in European Patent EP 1 207 875 are hardly suitable for the manufacture of parenteral forms of administration, either. The disadvantage of all the cited compounds is that the injection solutions made from then may be physiologically intolerable even after being diluted with, for example, 250-500 ml 5% glucose solution, and they are intravenously administered unphysiological components having an unknown toxicological profile. All hydroxy derivatives whose use was proposed in European Patent EP 1 207 875 for preparing oxaliplatin solutions are not among the standard adjuvants having known side-effects that are used in injectables. Normally, these compounds are only used as adjuvants in pharmaceutical preparations for external and oral administration.

The task of the present invention is to present stable, ready-to-use oxaliplatin solution concentrates without the disadvantages of the related-art solutions having carbohydrate additives, and the agent does not tend to spontaneously precipitate or crystallize even when the oxaliplatin concentration is above 6 mg/ml. Another essential task of the invention is to present advantageous methods for producing such solutions.

### SUMMARY OF THE INVENTION

One embodiment of the invention is directed to a pharmaceutical composition comprising a solution of oxaliplatin and a physiologically-compatible carbohydrate in water.

In a preferred embodiment, the physiologically-compatible carbohydrate is selected from the group consisting of glucose, maltose, fructose, trehalose, galactose, dextran, and mixtures thereof.

In a preferred embodiment, the pharmaceutical composition comprising a solution of oxaliplatin and a physiologically-compatible carbohydrate in water further comprises additional tonic, pH adjusting, buffering, or preserving adjuvants.

In a preferred embodiment, the pharmaceutical composition comprising a solution of oxaliplatin and a physiologically-compatible carbohydrate in water has a oxaliplatin concentration ranging from about 5 mg/ml to about 25 mg/ml. More preferably, the oxaliplatin concentration ranges from about 8 mg/ml to about 20 mg/ml.

In a preferred embodiment, the pharmaceutical composition comprising a solution of oxaliplatin and a physiologically-compatible carbohydrate in water has a oxaliplatin concentration higher than the maximum soluble concentration in pure water.

In a preferred embodiment, the pharmaceutical composition comprising a solution of oxaliplatin and a physiologically-compatible carbohydrate in water has a carbohydrate concentration of at least about 50 mg/ml of water.

In a preferred embodiment, the pharmaceutical composition comprising a solution of oxaliplatin and a physiologically-compatible carbohydrate in water is not reconstituted from a solid oxaliplatin preparation immediately before being administered to a mammal.

Another aspect of the invention is directed towards a method of treating tumor-related illnesses comprising administering the pharmaceutical composition comprising a solution of oxaliplatin and a physiologically-compatible carbohydrate in water to a mammal in need thereof.

Another aspect of the invention is directed towards a method of preparing the pharmaceutical composition comprising a solution of oxaliplatin and a physiologically-compatible carbohydrate in water, wherein oxaliplatin is dissolved in a carbohydrate-containing solvent at a temperature of from about 2° C. to about 15° C.

Another embodiment of the invention is directed towards a pharmaceutical composition comprising a solution of oxaliplatin and more than about 50 mg/ml glucose in water.

In a preferred embodiment, the pharmaceutical composition comprising a solution of oxaliplatin and more than about 50 mg/ml glucose in water has a oxaliplatin concentration of at least about 10 mg/ml.

In a preferred embodiment, the pharmaceutical composition comprising a solution of oxaliplatin and more than about 50 mg/ml glucose in water further comprises an acid, wherein the anion of the acid does not impair the stability of the solution.

Preferably, the acid is selected from the group consisting of phosphoric acid, sulfuric acid, methane sulfonic acid, ethane sulfonic acid, para-toluenesulfonic acid, and mixtures thereof.

In a preferred embodiment, the pharmaceutical composition comprising a solution of oxaliplatin and more than about 50 mg/ml glucose in water is not reconstituted from a solid oxaliplatin preparation immediately before being administered to a mammal.

Another aspect of the invention is directed towards a method of treating tumor-related illnesses comprising administering the pharmaceutical composition comprising a solution of oxaliplatin and more than about 50 mg/ml glucose in water to a mammal in need thereof.

Another aspect of the invention is directed towards a method of preparing a pharmaceutically acceptable solution of oxaliplatin in water comprising adding a physiologically-compatible carbohydrate to the solution, wherein the oxaliplatin does not precipitate or crystallize when the oxaliplatin concentration is more than about 6 mg/ml.

In a preferred embodiment, the oxaliplatin does not precipitate or crystallize when the oxaliplatin concentration is from about 7 mg/ml to about 8 mg/ml.

Another aspect of the invention is directed towards a method of improving the solubility of oxaliplatin in aqueous solutions comprising adding a physiologically-compatible carbohydrate to the solution.

### DETAILED DESCRIPTION OF THE INVENTION

Unless otherwise indicated, the percentages in the present application refer to percent by weight (weight %). The term “weight volume” refers to the standard definition known in the art (g/L).

Experiments showed that the oxaliplatin concentrates according to invention are stable against crystallization and spontaneous precipitation of the agent, and the oxaliplatin stability is even drastically improved in these concentrates. For example, only minor decomposition occurs in oxaliplatin solutions at a concentration of 10 mg/ml in 20% -82% weight volume aqueous glucose solutions (corresponding to 22% -90% weight volume glucose monohydrate) in which a pH of approximately 5.5 arises without additional additives. In particular, these investigations showed that the formation of undesirable diamino DACH platinum (II) dimer (III) may be reduced or suppressed by increasing the glucose concentration.

The agent oxaliplatin is relatively insoluble in water at approximately 7.5 mg/ml, and it dissolves very slowly to form a clear, visibly particle-free solution under conventional conditions. This may only be accelerated by heat (40° C.), which in turn promotes the breakdown of the agent. Another task of invention is therefore to present a method that allows the agent to quickly dissolve under particularly gentle conditions, especially without being heated or at low temperatures.

Experiments indicated that (see Tables 1 and 2) it is particularly suitable to use carbohydrate-containing solutions as the solvent to prepare the concentrate according to the invention with oxaliplatin concentrations of from about 5 mg/ml to about 50 mg/ml.

The solvents in the solutions are preferably physiologically-compatible polysaccharides, oligosaccharides, trisaccharides, disaccharides, and monosaccharides, more preferably monosaccharides, and even more preferably glucose.

The concentration of the carbohydrate in the concentrates may be adapted to the desired oxaliplatin concentration and the utilized carbohydrate. For example, depending on the carbohydrate, its solubility and the desired oxaliplatin concentration, carbohydrate concentrations ranging from about 20 mg/ml to about 1000 mg/ml may be used. A gentle procedure, such as at low temperatures, should be used in the aseptic production of the solutions (dissolving, filtering, filling, and intermediate storage). Table 1: Dissolving speed of oxaliplatin for obtaining a concentration of 5 mg/ml in water and a 5% glucose solution.

TABLE 1
Dissolving speed of oxaliplatin for obtaining a concentration
of 5 mg/ml in water and a 5% glucose solution.

| Test solution | | | |
|---|---|---|---|
| (pH not adjusted) | 40° C. | 20-25° C. | 10-15° C. |
| Oxaliplatin (c = 5 mg/ml) | <15 min | <60 min | <420 min |
| in water for injection. | | | |
| Oxaliplatin (c = 5 mg/ml) | <15 min | <60 min | <360 min |
| in 5% glucose | | | |

TABLE 2
Dissolving speed of oxaliplatin to obtain a concentration
of 10 mg/ml in water and glucose solutions of different
concentrations.

| Test solution (pH not adjusted) | 40° C. | 20-25° C. | 10-15° C. |
|---|---|---|---|
| Oxaliplatin (c = 10 mg/ml) | <60 min | not possible | not possible |
| in water for injection | | | |
| Oxaliplatin (c = 10 mg/ml) | <30 min | <90 min | <420 min |
| in 10% glucose | | | |
| Oxaliplatin (c = 10 mg/ml) | <15 min | <75 min | <270 min |
| in 20% glucose | | | |
| Oxaliplatin (c = 10 mg/ml) | <15 min | <60 min | <240 min |
| in 30% glucose | | | |
| Oxaliplatin (c = 10 mg/ml) | <15 min | <45 min | <180 min |
| in 40% glucose | | | |
| Oxaliplatin (c = 10 mg/ml) | <15 min | <45 min | <150 min |
| in 50% glucose | | | |

As shown in Tables 1 and 2, adding a carbohydrate such as glucose improves the solubility. Whereas oxaliplatin concentrations of 10 mg/ml in solutions without carbohydrates may only be attained by significantly raising the temperature, it is easy to dissolve this quantity of agent in glucose solutions at relatively low temperatures. In addition, the dissolving speed improves drastically by adding carbohydrates, which makes it substantially easier to produce the solutions.

In contrast, changing the pH of the solutions from a range of from about 5.0 to about 6.5, to a range of from about 3.0 to about 6.0 using oxalic acid, phosphoric acid or sulfuric acid does not shorten the dissolving time. The results clearly illustrate that adding the carbohydrates when producing the concentrated solutions having an unadjusted pH increases the dissolving speed of oxaliplatin, especially at much lower temperatures, which in turn helps keep the agent from breaking down. According to the invention, a temperature range of from about 10° C. to about 25° C. is preferable for gently dissolving oxaliplatin. A temperature of from about 10° C. to about 20° C. is more preferable, and a temperature of from about 10° C. to about 15° C. is even more preferable.

The invention's solution to the above-described the task is to stabilize oxaliplatin solutions by adding a carbohydrate preferably selected from the group comprising glucose, galactose, fructose, maltose, lactose and dextran (10-70), more preferably lactose, glucose and/or galactose. The preferred concentration of the carbohydrate in such solutions is preferably from about 10 weight percent to about 100 weight percent, and more preferably from about 10 weight percent to about 50 weight percent.

In addition to increasing the solubility of oxaliplatin and hence stabilizing the solution, adding the aforementioned carbohydrates to the solutions according to the invention also allow higher concentrations of oxaliplatin without the agent undesirably precipitating or crystallizing. In addition, the carbohydrate additives according to the invention help lowering the hydroxide ion concentration since the compounds are capable of lowering the pH of the solution, as illustrated by the summary of the investigative results in Table 3.

TABLE 3
Relationship of the pH of aqueous glucose
solutions to the glucose concentration

| | Experimentally determined pH |
|---|---|
| Glucose | of glucose dissolved in |
| Concentration | water (20-25° C.) |
| 5% glucose | pH 6.2 |
| 10% glucose | pH 6.1 |
| 20% glucose | pH 5.8 |
| 30% glucose | pH 5.7 |
| 40% glucose | pH 5.5 |
| 50% glucose | pH 5.3 |

The solutions according to the invention may be stabilized with or without adding an acid. When the agent concentrations are more than about 5 mg/ml, a carbohydrate should be added to improve solubility. In a preferred embodiment, the solution concentrate according to the invention comprises about 10 mg/ml oxaliplatin, about 200 mg/ml glucose monohydrate, and enough phosphoric acid to adjust the pH to approximately 3.5.

As described at the onset, the recommended dose in oxaliplatin therapy is 85 mg/m² body surface. To date, only solutions reconstituted from a lyophilizate containing 5 mg/ml have been available. Given the average dose of 150 mg oxaliplatin/1.8 m² body surface, this means diluting a reconstituted solution of 30 ml with 250 ml to 500 ml 5% glucose solution. For reasons of expense, it would be advantageous to use small packages to store the drugs (such as injection vials). To lower expenses and ensure safe handling, it is also advantageous to use small volumes of solution. The ready-to-use infusion solution concentrates according to the invention have these additional advantages.

In the experiments carried out for the present invention, it was shown that oxaliplatin is soluble in an aqueous solution at maximum concentrations of 7 to 8 mg/ml (at 25° C., Table 4, first entry). By using additional tonic, pH-adjusting, buffering or preserving adjuvants, which are also dissolved, a maximum concentration of 7 mg/ml oxaliplatin in water is attained. This low solubility with the additional hazard of undesirable crystallization in supersaturated solutions at storage temperatures lower than 25° C. means that oxaliplatin's maximum concentration for pharmaceutical use is about 5 mg/ml. In order to gain the aforementioned additional advantages of an infusion solution concentrate with, for example, 10 mg oxaliplatin/ml water, the solubility must be increased.

The following examples illustrate the suitability of selected carbohydrates and concentrations according to the invention.

### EXAMPLES

The concentrated solutions according to the invention are prepared in the usual manner, preferably with a very short processing time (dwell time), while continuously maintaining a very cool dissolving temperature that does not degrade the agent. Approximately 80% of the required solvent (water for injection purposes) is added to the mixing vessel, and the solublizing adjuvant (such as glucose monohydrate) is dissolved at a temperature of from about 20° C. to about 25° C. Then the original solutions are cooled to a temperature of from about 10° C. to about 15° C. Other additives and/or adjuvant such as antioxidants, additional solubilizers, solvents, preservatives, etc. may be used. The pH may also be set by acids, bases are buffers in this step.

After preparing the solvent according to the invention, the appropriate amount of oxaliplatin is added and dissolved in the batch solution. Water (preferably cooled to a temperature of from about 10° C. to about 15° C.) for injection purposes is added to the batches to the desired end volume, and the solutions obtained in this manner are also sterile-filtered at low temperatures (preferably at temperature of from about 10° C. to about 15° C.) into a sterile receiver. The solution is aseptically poured into suitable sterile, tightly-sealable containers such as injection vials having injection or infusion plugs.

The ready-to-use solution concentrates are preferably stored at a temperature of from about 2° C. to about 8° C.

Results demonstrated that using aqueous carbohydrate solutions, particularly glucose solutions, improves the solubility of the agent oxaliplatin enough for the agent to be dissolved at low temperatures. The medication may hence be prepared at low temperatures, i.e. more gently than the known related art. In addition, the solubility of the agent is improved enough so that the solutions according to the invention represent oxaliplatin solution concentrates that are much easier and less hazardous to use than the known related-art lyophilizates, and they enable long-term storage in economical, small containers. The oxaliplatin solution concentrates according to the invention manifest a completely unexpected high long-term stability without pharmaceutically problematic additives.

### Example 1

### Improving Solubility by Adding Carbohydrate

In experiments to increase the solubility of oxaliplatin in an aqueous solution by adding large amounts of glucose or other carbohydrates (such as maltose, fructose, galactose, extra and 10, dextran 70 and dextran 40) at high concentrations (from 20% to 50%), it was discovered that concentrated oxaliplatin solutions can contain far more than 10 mg/ml. Such concentrates are also stable at a temperature of from about 2° C. to about 8° C. over long periods without forming crystals. In these initial experiments, glucose solutions were used that contained 20%, 30% and 40% glucose (corresponding to 22%, 33% or 44% glucose monohydrate) with an uninfluenced pH (see Table 4).

Table 4 compiles the results of the experiments carried out in this context. The saturated solution supernatant was produced and prepared for analysis as described in European Patent EP 1 207 875. The pH was not adjusted in the dissolving experiments (the pH remained uninfluenced). The amounts were determined via HPLC.

TABLE 4
Relationship of the solubility of oxaliplatin in aqueous
solutionsto the added carbohydrates and their
concentration in the solution.

| | | Oxaliplatin | pH of the |
|---|---|---|---|
| Amount of | | concentration of the | saturated |
| carbohydrate | Carbohydrate | saturated solution | solution |
| 0% | Glucose | 7.66 mg/ml | pH 6.1 |
| 5% | Glucose | 8.14 mg/ml | pH 5.9 |
| 10% | Glucose | 10.74 mg/ml | pH 5.8 |
| 20% | Glucose | 11.85 mg/ml | pH 5.7 |
| 30% | Glucose | 12.66 mg/ml | pH 5.6 |
| 40% | Glucose | 13.54 mg/ml | pH 5.6 |
| 50% | Glucose | 14.23 mg/ml | pH 5.4 |
| 50% | Maltose | 17.83 mg/ml | pH 3.7 |
| 50% | Fructose | 12.56 mg/ml | pH 4.7 |
| 25% | Galactose | 11.53 mg/ml | pH 6.3 |
| 25% | Dextran 10 | 10.90 mg/ml | pH 4.8 |
| 25% | Dextran 70 | 10.90 mg/ml | pH 4.1 |
| 25% | Dextran 40 | 10.85 mg/ml | pH 4.2 |
| 10% | Lactose | 10.12 mg/ml | pH 4.3 |
| 10% | Trehalose | 10.32 mg/ml | pH 4.6 |

In the experiments, lactose, trehalose, glucose, maltose, fructose, galactose, dextranes (10 to 70) and dimethylisosorbital proved to be particularly suitable solubilizers in obtaining oxaliplatin concentrations above about 7 mg/ml oxaliplatin.

In the experiments for determining the suitability and maximum solubility of oxaliplatin in hydroxyl-derivative-containing aqueous solutions, glucose, maltose, fructose, galactose, dextran 10, dextran 40, dextran 70 and different polyethylene glycols such as polyethylene glycol 200, 300, 400 and 600 proved to be particularly suitable adjuvants for obtaining oxaliplatin concentrations of more than about 7 mg/ml solvent.

Although the aforementioned oxaliplatin solution concentrates already show sufficient stability of the agent and solution, the pH of the carbohydrate solutions according to the invention may be adjusted to the optimum stability by adding acids and/or buffers, as it is still above approximately pH 5.0 in the directly-obtained solutions.

To determine the agent stability of the concentrates according to the invention, the decomposition products were determined over time. The results of these measurements are summarized in the following examples.

### Example 2

### Stability of the Oxaliplatin Solution Concentrates According to the Invention

To examine the stability of the solution concentrates according to the invention, glucose solutions of different concentrations as solvents were added to corresponding oxaliplatin solutions (c =10 mg/ml). The agent was dissolved while heating the solution to 40-45° C. over 30 min. The solutions obtained in this manner were stored protected from light at 25 and 60° C., and the amount of platinum-containing decomposition products II and III of oxaliplatin was determined via HPLC. The amount of the decomposition product oxalic acid was determined from this data. The amount of decomposition products in the solutions according to the invention are shown over time for the two storage temperatures in Table 5.

TABLE 5
Stability of the oxaliplatin solution concentrates under
accelerated conditions; pH not adjusted (pH = 5.6 ± 0.4).
Decomposition products determined via HPLC.

| Test solution | | | | |
|---|---|---|---|---|
| conc. | | | | Diaquo DACH |
| oxaliplatin | | Oxalic | Diaquo DACH | platinum (II)- |
| solvent | Test cycle | acid | platinum (II) (II) | Dimer (III) |
| c = 10 mg/ml | Start^{a} | 0.242% | 0.117% | not det. |
| 10% glucose | 1d-25° C. | 0.675% | 0.283% | <0.01% |
| | 1d-60° C. | 0.795% | 0.275% | 0.049% |
| c = 10 mg/ml | Start^{a} | 0.200% | 0.110% | not det. |
| 20% glucose | 1d-25° C. | 0.618% | 0.300% | <0.01% |
| | 1d-60° C. | 0.779% | 0.255% | 0.029% |
| c = 10 mg/ml | Start^{a} | 0.234% | 0.117% | not det. |
| 30% glucose | 1d-25° C. | 0.646% | 0.300% | <0.01% |
| | 1d-60° C. | 0.799% | 0.273% | <0.01% |
| c = 10 mg/ml | Start^{a} | 0.212% | 0.125% | not det. |
| 40% glucose | 1d-25° C. | 0.432% | 0.294% | <0.01% |
| | 1d-60° C. | 0.806% | 0.246% | <0.01% |
| c = 10 mg/ml | Start^{a} | 0.193% | 0.119% | not det. |
| 50% glucose | 1d-25° C. | 0.572% | 0.292% | not det. |
| | 1d-60° C. | 0.648% | 0.234% | not det. |

^{a}The solutions were prepared while being heated to 40-45° C. over 30 min and immediately stored at 20-25° C. and 60° C.

Table 5 shows that the agent substantially decomposes when the solution is dissolved at a high temperature required by the related art in order to obtain concentrated oxaliplatin solutions (for example c=5 mg/ml without adding solubilizers). This decomposition increases over time at higher temperatures. However, addition of a carbohydrate (glucose in this instance) reduces such decomposition, especially at very high concentrations.

TABLE 6
Long-term stability of oxaliplatin solution concentrates
at reduced temperatures; pH not adjusted
(pH = 5.6 ± 0.2). Decomposition products
determined via HPLC.

| | | | | Diaquo |
|---|---|---|---|---|
| Test solution | | | Diaquo | DACH |
| conc. | | | DACH | platinum |
| oxaliplatin | | Oxalic | platinum | (II)-Dimer |
| solvent | Test cycle | acid | (II) (II) | (III) |
| c = 10 mg/ml | Start^{a} | 0.21% | 0.105% | <0.01% |
| 20% glucose | 12 mo.-(2-8° C.) | 0.32% | 0.152% | <0.029% |
| | Start^{b} | 0.16% | 0.060% | <0.01% |
| | 12 mo.-(2-8° C.) | 0.22% | 0.083% | <0.01% |
| c = 10 mg/ml | Start^{a} | 0.20% | 0.101% | <0.01% |
| 30% glucose | 12 mo.-(2-8° C.) | 0.33% | 0.145% | <0.01% |
| | Start^{b} | 0.19% | 0.072% | <0.01% |
| | 12 mo.-(2-8° C.) | 0.26% | 0.098% | <0.01% |
| c = 10 mg/ml | Start^{a} | 0.22% | 0.103% | <0.01% |
| 40% glucose | 12 mo.-(2-8° C.) | 0.31% | 0.146% | <0.01% |
| | Start^{b} | 0.17% | 0.064% | <0.01% |
| | 12 mo.-(2-8° C.) | 0.29% | 0.091% | <0.01% |
| c = 10 mg/ml | Start^{a} | 0.20% | 0.106% | <0.01% |
| 50% glucose | 12 mo.-(2-8° C.) | 0.30% | 0.150% | <0.01% |
| | Start^{a} | 0.20% | 0.060% | <0.01% |
| | 12 mo.-(2-8° C.) | 0.28% | 0.109% | <0.01% |

^{a}The solutions were produced at 20-25° C. and immediately stored at 2-8° C.
^{b}The solutions were produced at 10-15° C. and immediately stored at 2-8° C.

Time-resolved measurements (results summarized in Table 6) of correspondingly prepared and stored test batches were taken to see if cool storage (2-8° C.) and the nondestructive production of the solution at low temperatures enabled by the present invention may yield long-term stable oxaliplatin solution concentrates.

The results summarized in Table 6 illustrate that using carbohydrate solutions (such as glucose monohydrate in water) as solvents to produce oxaliplatin solution concentrates enables the preparation of injection concentrates with long-term stability. Preparing these concentrates without heating them reduces the decomposition of oxaliplatin (which is otherwise very unstable in aqueous solutions) to such an extent that not even 0.1% of the decomposition product II and less than 0.01% of the dimers III were observed after being stored for a year.

To further stabilize the solution concentrates, it is desirable to add acids or buffers to reduce the pH and thus lower the concentration of aggressive hydroxide anions. The effect of a phosphoric acid additive was investigated as an example to test the assumption that adding an acid whose anion does not destabilize oxaliplatin to the oxaliplatin solution concentrates containing carbohydrate solutions as the solvent further reduces the decomposition of the agent. The decomposition products of oxaliplatin solution concentrates (C=10 mg/ml), with a pH adjusted with IN phosphoric acid to 3.8±0.2, were measured over time and quantitatively determined via HPLC under the same conditions as those under Table 6. The results are summarized in Table 7.

TABLE 7
Long-term stability of oxaliplatin solution concentrates at reduced
temperatures; pH preadjusted using 1 N phosphoric acid
(pH = 3.8 ± 0.2). The decomposition products were
determined via HPLC.

| Test solution | | | Diaquo | Diaquo |
|---|---|---|---|---|
| conc. | | | DACH | DACH |
| oxaliplatin | | Oxalic | platinum | platinum (II) |
| solvent | Test cycle | acid | (II) (II) | dimer (III) |
| c = 10 mg/ml | Start^{a} | 0.18% | 0.093% | <0.01% |
| 20% glucose | 12 mo.-(2-8° C.) | 0.28% | 0.141% | <0.020% |
| | Start^{b} | 0.14% | 0.052% | <0.01% |
| | 12 mo.-(2-8° C.) | 0.20% | 0.098% | <0.01% |
| c = 10 mg/ml | Start^{a} | 0.19% | 0.100% | <0.01% |
| 30% glucose | 12 mo.-(2-8° C.) | 0.30% | 0.138% | <0.01% |
| | Start^{b} | 0.13% | 0.052% | <0.01% |
| | 12 mo.-(2-8° C.) | 0.20% | 0.098% | <0.01% |
| c = 10 mg/ml | Start^{a} | 0.20% | 0.092% | <0.01% |
| 40% glucose | 12 mo.-(2-8° C.) | 0.28% | 0.126% | <0.01% |
| | Start^{b} | 0.14% | 0.054% | <0.01% |
| | 12 mo.-(2-8° C.) | 0.21% | 0.101% | <0.01% |
| c = 10 mg/ml | Start^{a} | 0.19% | 0.088% | <0.01% |
| 50% glucose | 12 mo.-(2-8° C.) | 0.25% | 0.111% | <0.01% |
| | Start^{b} | 0.16% | 0.058% | <0.01% |
| | 12 mo.-(2-8° C.) | 0.22% | 0.100% | <0.01% |

^{a}The solutions were prepared at 20-25° C. and immediately stored at2-8° C.
^{b}The solutions were prepared at 10-15° C. and immediately stored at 2-8° C.

Results summarized in Table 7 indicate that the solution concentrates may be further stabilized via reduction of the pH by adding an acid whose anion does not destabilize oxaliplatin.

The experiments summarized in Example 2 (Tables 5-7) clearly indicate that using carbohydrate solutions as the solvent in preparing oxaliplatin solution concentrates results in pharmaceutical preparations with long-term stability. Furthermore, using solvents according to the invention allows oxaliplatin solutions to be prepared in a particularly nondestructive manner. Finally, adding acids that do not destabilize the agent oxaliplatin (such as phosphoric acid or sulfuric acid) also has a positive effect on the concentrates.

### Examples 3-69

### Composition of Exemplary Oxaliplatin Solution Concentrates According to the Invention

Examples of oxalic acid solution concentrates according to the invention are summarized below.

TABLE 8
pH-adjusted oxaliplatin solution concentrates according to the invention

| | | | | Carbohydrate. | |
|---|---|---|---|---|---|
| | Oxaliplatin | | | Hydroxy | |
| No | (mg/ml) | pH | Acid | Derivate | Buffer Base |
| 3. | 7.5 | 3.2 | Phosphoric acid | | |
| 4. | 7.5 | 3.2 | Phosphoric acid | 10 mg/ml glucose | |
| | | | | monohydrate | |
| 5. | 8 | 3.2 | Phosphoric acid | 10 mg/ml glucose | |
| | | | | monohydrate | |
| 6. | 8 | 3.2 | Phosphoric acid | 10 mg/ml fructose | |
| 7. | 8 | 3.2 | Phosphoric acid | | |
| 8. | 8 | 3.2 | Sulfuric acid | | |
| 9. | 8 | 3.0 | Phosphoric acid | 10 mg/ml glucose | 5 mg/ml disodium |
| | | | | monohydrate, 100 mg/ml | hydrogen phosphate |
| | | | | polyethylene | |
| | | | | glycol 400 | |
| 10. | 8 | 3.3 | Phosphoric acid | 100 mg/ml | 2 mg/ml disodium |
| | | | | polyethylene | hydrogen phosphate |
| | | | | glycol 600 | |
| 11. | 8 | 3.5 | Lactic acid | 100 mg/ml | 2 mg/ml disodium |
| | | | | polyethylene | hydrogen phosphate |
| | | | | glycol 600 | |
| 12. | 8 | 3.5 | Phosphoric acid | 100 mg/ml lactose | |
| | | | | monohydrate | |
| 13. | 10 | 3.0 | Phosphoric acid | 100 mg/ml | |
| | | | | glucose | |
| | | | | monohydrate | |
| 14. | 10 | | | 150 mg/ml | |
| | | | | glucose | |
| | | | | monohydrate | |
| 15. | 10 | | | 200 mg/ml | |
| | | | | glucose | |
| | | | | monohydrate | |
| 16. | 10 | | | 250 mg/ml | |
| | | | | glucose | |
| | | | | monohydrate | |
| 17. | 10 | | | 300 mg/ml | |
| | | | | glucose | |
| | | | | monohydrate | |
| 18. | 10 | | | 350 mg/ml | |
| | | | | glucose | |
| | | | | monohydrate | |
| 19. | 10 | | | 400 mg/ml | |
| | | | | glucose | |
| | | | | monohydrate | |
| 20. | 10 | | | 450 mg/ml | |
| | | | | glucose | |
| | | | | monohydrate | |
| 21. | 12 | | | 500 mg/ml | |
| | | | | glucose | |
| | | | | monohydrate | |
| 22. | 12 | | | 600 mg/ml | |
| | | | | glucose | |
| | | | | monohydrate | |
| 23. | 12 | | | 700 mg/ml | |
| | | | | glucose | |
| | | | | monohydrate | |
| 24. | 15 | | | 800 mg/ml | |
| | | | | glucose | |
| | | | | monohydrate | |
| 25. | 20 | | | 900 mg/ml | |
| | | | | glucose | |
| | | | | monohydrate | |
| 26. | 10 | 3.0 | Phosphoric acid | 150 mg/ml | |
| | | | | glucose | |
| | | | | monohydrate | |
| 27. | 10 | 3.0 | Phosphoric acid | 200 mg/ml | |
| | | | | maltose | |
| | | | | monohydrate | |
| 28. | 10 | 3.2 | Phosphoric acid | 350 mg/ml | |
| | | | | maltose | |
| | | | | monohydrate | |
| 29. | 12 | 3.4 | Phosphoric acid | 500 mg/ml | |
| | | | | maltose | |
| | | | | monohydrate | |
| 30. | 10 | 3.0 | Phosphoric acid | 150 mg/ml | 2.5 mg/ml disodium |
| | | | | glucose | hydrogen phosphate |
| | | | | monohydrate | |
| 31. | 10 | 2.9 | Phosphoric acid | 200 mg/ml | |
| | | | | glucose | |
| | | | | monohydrate | |
| 32. | 10 | 3.2 | Phosphoric acid | 200 mg/ml | |
| | | | | glucose | |
| | | | | monohydrate | |
| 33. | 10 | 3.6 | Phosphoric acid | 200 mg/ml | |
| | | | | glucose | |
| | | | | monohydrate | |
| 34. | 10 | 3.8 | Phosphoric acid | 200 mg/ml | |
| | | | | glucose | |
| | | | | monohydrate | |
| 35. | 10 | 4.0 | Phosphoric acid | 200 mg/ml | |
| | | | | glucose | |
| | | | | monohydrate | |
| 36. | 10 | 4.2 | Phosphoric acid | 200 mg/ml | |
| | | | | glucose | |
| | | | | monohydrate | |
| 37. | 10 | 3.4 | Phosphoric acid | 200 mg/ml | 0.25 mg/ml disodium |
| | | | | glucose | hydrogen phosphate |
| | | | | monohydrate | |
| 38. | 10 | 3.6 | Phosphoric acid | 300 mg/ml | 0.25 mg/ml disodium |
| | | | | glucose | hydrogen phosphate |
| | | | | monohydrate | |
| 39. | 10 | 3.8 | Phosphoric acid | 400 mg/ml | 0.15 mg/ml disodium |
| | | | | glucose | hydrogen phosphate |
| | | | | monohydrate | |
| 40. | 10 | 4.0 | Phosphoric acid | 500 mg/ml | 0.25 mg/ml disodium |
| | | | | glucose | hydrogen phosphate |
| | | | | monohydrate | |
| 41. | 10 | 3.2 | Sulfuric acid | 200 mg/ml | |
| | | | | glucose | |
| | | | | monohydrate | |
| 42. | 10 | 3.4 | Sulfuric acid | 200 mg/ml | 0.25 mg/ml disodium |
| | | | | glucose | hydrogen phosphate |
| | | | | monohydrate | |
| 43. | 10 | 3.6 | Sulfuric acid | 300 mg/ml | 0.25 mg/ml disodium |
| | | | | glucose | hydrogen phosphate |
| | | | | monohydrate | |
| 44. | 10 | 3.8 | Sulfuric acid | 400 mg/ml | 0.25 mg/ml disodium |
| | | | | glucose | hydrogen phosphate |
| | | | | monohydrate | |
| 45. | 10 | 4.0 | Sulfuric acid | 500 mg/ml | 25 mg/ml disodium |
| | | | | glucose | hydrogen phosphate |
| | | | | monohydrate | |
| 46. | 10 | 3.4 | Phosphoric acid | 500 mg/ml | |
| | | | | glucose | |
| | | | | monohydrate | |
| 47. | 10 | 3.8 | Sulfuric acid | 800 mg/ml | |
| | | | | glucose | |
| | | | | monohydrate | |
| 48. | 10 | 3.9 | Ethane sulfonic | 900 mg/ml | |
| | | | acid | glucose | |
| | | | | monohydrate | |
| 49. | 10 | 3.5 | Lactic acid | 200 mg/ml | |
| | | | | glucose | |
| | | | | monohydrate | |
| 50. | 10 | 3.5 | Lactic acid | 200 mg/ml | 1 mg/ml sodium lactate |
| | | | | glucose | |
| | | | | monohydrate | |
| 51. | 10 | 3.5 | Lactic acid | 250 mg/ml | 20 mg/ml sodium |
| | | | | glucose | lactate |
| | | | | monohydrate | |
| 52. | 10 | 3.5 | Lactic acid | 300 mg/ml | 2 mg/ml sodium lactate |
| | | | | glucose | |
| | | | | monohydrate | |
| 53. | 10 | 4.0 | Lactic acid | 400 mg/ml | 2 mg/ml sodium lactate |
| | | | | glucose | |
| | | | | monohydrate | |
| 54. | 10 | 3.5 | Phosphoric acid | 200 mg/ml | |
| | | | | glucose | |
| | | | | monohydrate | |
| 55. | 10 | 3.5 | Phosphoric acid | 500 mg/ml | |
| | | | | glucose | |
| | | | | monohydrate | |
| 56. | 10 | 3.5 | Phosphoric acid | 800 mg/ml | |
| | | | | glucose | |
| | | | | monohydrate | |
| 57. | 10 | 3.5 | Phosphoric acid | 900 mg/ml | |
| | | | | glucose | |
| | | | | monohydrate | |
| 58. | 10 | 6.0 | Phosphoric acid | 100 mg/ml | |
| | | | | glucose | |
| | | | | monohydrate | |
| 59. | 10 | 6.0 | Phosphoric acid | 200 mg/ml | |
| | | | | glucose | |
| | | | | monohydrate | |
| 60. | 10 | 6.0 | Phosphoric acid | 500 mg/ml | |
| | | | | glucose | |
| | | | | monohydrate | |
| 61. | 10 | 6.0 | Phosphoric acid | 800 mg/ml | |
| | | | | glucose | |
| | | | | monohydrate | |
| 62. | 15 | 2.9 | Phosphoric acid | 900 mg/ml | |
| | | | | glucose | |
| | | | | monohydrate | |
| 63. | 15 | 6.0 | Phosphoric acid | 900 mg/ml | |
| | | | | glucose | |
| | | | | monohydrate | |
| 64. | 15 | 6.0 | Phosphoric acid | 900 mg/ml | |
| | | | | glucose | |
| | | | | monohydrate | |
| 65. | 20 | 3.3 | Phosphoric acid | 900 mg/ml | |
| | | | | glucose | |
| | | | | monohydrate | |
| 66. | 20 | 3.4 | Phosphoric acid | 700 mg/ml | |
| | | | | glucose | |
| | | | | monohydrate | |
| 67. | 20 | 3.4 | Phosphoric acid | 700 mg/ml | |
| | | | | fructose | |
| | | | | monohydrate | |
| 68. | 15 | 3.4 | Phosphoric acid | 500 mg/ml | |
| | | | | fructose | |
| | | | | monohydrate | |
| 69. | 25 | 3.0 | Phosphoric acid | 900 mg/ml | |
| | | | | glucose | |
| | | | | monohydrate | |

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to, limit its scope in any way. The examples do not include detailed descriptions of conventional methods.

What is claimed is:

1. A pharmaceutical composition comprising a solution of oxaliplatin and a physiologically-compatible carbohydrate in water.

2. The composition according to claim 1, wherein the physiologically-compatible carbohydrate is selected from the group consisting of glucose, maltose, fructose, trehalose, galactose, dextran, and mixtures thereof.

3. The composition according to claim 1, further comprising additional tonic, pH adjusting, buffering, or preserving adjuvants.

4. The composition according to claim 1, wherein the oxaliplatin concentration ranges from about 5 mg/ml to about 25 mg/ml.

5. The composition according to claim 1, wherein the oxaliplatin concentration ranges from about 8 mg/ml to about 20 mg/ml.

6. The composition according to claim 1, wherein the oxaliplatin concentration is higher than the maximum soluble concentration in pure water.

7. The composition according to claim 1, wherein the carbohydrate concentration is at least about 50 mg/ml of water.

8. The composition according to claim 1, wherein the composition is not reconstituted from a solid oxaliplatin preparation immediately before being administered to a mammal.

9. A method of treating tumor-related illnesses comprising administering the pharmaceutical composition according to claim 1 to a mammal in need thereof.

10. A method of preparing the pharmaceutical composition according to claim 1, wherein oxaliplatin is dissolved in a carbohydrate-containing solvent at a temperature of from about 2° C. to about 15° C.

11. A pharmaceutical composition comprising a solution of oxaliplatin and more than about 50 mg/ml glucose in water.

12. The composition according to claim 11, wherein the oxaliplatin concentration is at least about 10 mg/ml.

13. The composition according to claim 11, further comprising an acid, wherein the anion of the acid does not impair the stability of the solution.

14. The composition according to claim 13, wherein the acid is selected from the group consisting of phosphoric acid, sulfuric acid, methane sulfonic acid, ethane sulfonic acid, para-toluenesulfonic acid, and mixtures thereof.

15. The composition according to claim 11, wherein the composition is not reconstituted from a solid oxaliplatin preparation immediately before being administered to a mammal.

16. A method of treating tumor-related illnesses comprising administering the pharmaceutical composition according to claim 11 to a mammal in need thereof.

17. A method of preparing a pharmaceutically acceptable solution of oxaliplatin in water comprising adding a physiologically-compatible carbohydrate to the solution, wherein the oxaliplatin does not precipitate or crystallize when the oxaliplatin concentration is more than about 6 mg/ml.

18. The method according to claim 17, wherein the oxaliplatin does not precipitate or crystallize when the oxaliplatin concentration-is from about 7 mg/ml to about 8 mg/ml.

19. A method of improving the solubility of oxaliplatin in aqueous solutions comprising adding a physiologically-compatible carbohydrate to the solution.
